(19) United States
(12) Patent Application Publication
(10) Pub. No.: US 2023/0167172 A1
(43) Pub. Date: Jun. 1, 2023

(54) PREVENTION OF VISIBLE PARTICLE FORMATION IN PARENTERAL PROTEIN SOLUTIONS

(71) Applicant: Hoffmann-La Roche Inc., Little Falls, NJ (US)

(72) Inventors: Keijiro HATADE, Tokyo (JP); Stefan Josef FISCHER, Basel (CH); Christina Roswitha HAEUSER, Basel (CH); Stephen Alfred HYLAND, Lörrach (DE); Tarik Ali KHAN, Basel (CH); Ralph LOVIS, Windisch (CH); Satya Krishna Kishore RAVURI, Pfeffingen (CH); Hitoshi SASAKI, Tokyo (JP)

(73) Assignee: Hoffmann-La Roche Inc., Little Falls, NJ (US)

(21) Appl. No.: 18/054,244

(22) Filed: Nov. 10, 2022

Related U.S. Application Data
(63) Continuation of application No. PCT/EP2021/062579, filed on May 12, 2021.

(30) Foreign Application Priority Data
May 15, 2020 (EP) 20174854.8

(51) Int. Cl. C07K 16/24

(52) U.S. Cl. CPC C07K 16/248

(57) ABSTRACT

The present invention provides methods to prevent the formation of visible particles in aqueous protein formulations, as well as compositions and pharmaceutical products obtained with said method.

### CROSS REFERENCE TO RELATED APPLICATIONS

This application is a continuation of International Application No. PCT/EP2021/062579, filed May 12, 2021, which claims priority to European Patent Application No. 20174854.8, filed May 15, 2020, which are incorporated herein by reference in its entirety.

The present invention relates to the field of aqueous protein compositions, in particular pharmaceutical antibody formulations for parenteral application, which are stabilized against the formation of visible particles.

### BACKGROUND OF THE INVENTION

Surfactants are crucial excipients in protein formulations as they protect the labile protein from interfacial stress that may lead to protein aggregation. Proteins, such as monoclonal antibodies (mAb), are administered parenterally, and only a limited choice of the surfactant is today approved for use in such formulations. Among the surfactants approved for use in parenteral antibody preparations are polysorbates (PS20, PS80) and poloxamer 188 (see Kishore R S. Part II: Challenges with Excipients—Polysorbate Degradation and Quality, in Challenges in Protein Product Development. AAPS Advances in the Pharmaceutical Sciences Series 38. Warne N and Mahler H C eds. Springer 2018, Switzerland. pp. 25-62). However, polysorbates (PS20 and PS80) can degrade over the shelf-life of a product either by oxidative degradation or by enzymatic, hydrolytic degradation. In particular, the latter yields free fatty acids (FFA) as degradation products, which can precipitate in solution and subsequently form sub-visible and visible particles (Tarik A Khan et. al., Protein-Polydimethylsiloxane Particles in Liquid Vial Monoclonal Antibody Formulations Containing Poloxamer 188; J. Pharmaceutical Sciences, published online Mar. 16, 2020).

There is thus a need to provide efficient solutions to prevent the formation of visible particles in aqueous protein solutions, in particular those using polysorbates as surfactants, and for long term storage. In accordance with the present invention it has been found that specific grades of PS80 provide long term stability of aqueous protein formulations.

### SUMMARY OF THE INVENTION

In one embodiment, the present invention provides aqueous protein formulations comprising PS80 (98), as defined herein, as surfactant.

In another embodiment, the PS80 (98) is present in said aqueous protein formulations in the range from 0.01 to 1% (w/v); or from 0.01 to 0.06% (w/v); or from 0.02 to 0.05% (w/v); or at about 0.02% (w/v).

In another embodiment, the present invention provides the use of PS80 (98) in the manufacture of aqueous protein formulations.

In yet another embodiment, the present invention provides the use of PS80 (98) to prevent the formation of visible particles in aqueous protein formulations.

### DETAILED DESCRIPTION OF THE INVENTION

Formation of visible particles consisting of free fatty acids (FFA) as a result of surfactant degradation, especially from polysorbate (PS20 and/or PS80) degradation, represents a major challenge in the biopharmaceutical industry as there is limited choice for surfactants in parenteral protein formulations such as, for example, parenteral preparations of therapeutic antibodies. Reducing or even eliminating visible particle formation due to polysorbate degradation, and thus the release of FFA, by various means is key for maintaining the quality of a parenteral pharmaceutical product over the time of its authorized shelf life.

Commercially available polysorbates (PS20 and 80) are chemically diverse mixtures containing mainly sorbitan POE fatty acid esters. The main species of PS80 contains a sorbitan head group with 4 chains of polyoxyethylene (POE) extending from it. Theoretically, there are a total of twenty POE units which are attached to each head group, although in practice, there may end up being more or less. Typically, there is a Gaussian-like distribution in the number of POE units, resulting in a heterogeneous mixture. Of the four POE groups attached to the sorbitan head groups, 1 to 3 of them are esterified to fatty acids (FAs) at their ends which can also terminate in a primary alcohol. The FAs found in PS80 are 14 to 18 carbons long and can have up to 3 double bonds along the chain. The most abundant FA is oleic acid (≥58%, 18 carbons, 1 double bond), followed by linoleic (18%, 18 carbons, 2 double bonds). The number of FA substitutions on an individual sorbitan head group can range from zero to 4. PS80 also has isosorbide head groups with zero to 2 FA substitutions. There also exists a significant amount of POE-FAs unattached to the head groups. (Journal of Pharmaceutical Sciences 109 (2020) 633-639)

The present inventors surprisingly found that the use of PS80 with an oleic acid content of at least 98%, relative to other fatty acids present, provides long term stability against the formation of visible particles in aqueous, parenteral protein formulations

PS80s of different grades from two manufacturers, SEPPIC and NOF Corporation (NOF), were compared for their ability to prevent visible particle formation in a commercial antibody preparation:

PS80, Montanox 80 PPI (SEPPIC)

PS80, HX2 (NOF)

PS80 (NOF) is the PS80 with at least 98% oleic acid in accordance with the present invention. It is also designated “PS80 (98)” herein.

Therefore, in one embodiment the present invention provides a stable aqueous composition comprising a protein together with pharmaceutically acceptable excipients such as, for example, buffers, stabilizers including antioxidants, and PS80 (98).

In one embodiment PS80 (98) is present in a concentration from 0.01 to 1% (w/v); or from 0.01 to 0.06% (w/v); or from 0.02 to 0.05% (w/v); or at about 0.02% (w/v).

In another embodiment, there is provided the composition as defined above, wherein the pH of said composition is in the range of 5 to 7. In one aspect the pH is about 6.

In another embodiment, the present invention provides a composition as defined herein before, wherein the protein is an antibody. In one aspect, the antibody is a monoclonal antibody. In another aspect the antibody is a human or humanized monoclonal, mono- or bispecific antibody.

In another embodiment, the present invention provides a composition as defined herein before, consisting of the following ingredients: 180 mg/mL tocilizumab, 20 mmol/L L-histidine/L-histidine hydrochloride monohydrate, 100 mmol/L LArginine/L-Arginine hydrochloride, 30 mmol/L L-methionine, 0.2 mg/mL PS80 (98); at pH=6.0. The antibody with the INN tocilizumab is known to the skilled person and is, for example, commercially available under the tradename ACTEMRA®. Tocilizumab is, for example, also disclosed in WO92/019759 and K. Sato et. al., Cancer Res 53(4), 1993; 851-856. The sequence information for tocilizumab can also be found under CAS Registry Number: 375823-41-9.

In another embodiment, the present invention provides the use of PS80 (98), as defined herein, for the manufacture of medicaments, especially for the manufacture of parenteral protein-, more specifically parenteral antibody preparations. In one embodiment the parenteral preparation is for subcutaneous (sc) application. In another embodiment, the parenteral preparation is for intravenous (iv) application.

In another embodiment, the present invention provides the use of PS80 (98), as defined herein, to prevent the formation of visible particles in parenteral protein, especially antibody preparations. In one embodiment the parenteral preparation is for subcutaneous (sc) application. In another embodiment, the parenteral preparation is for intravenous (iv) application.

The present parenteral protein- or antibody preparation are “stable”, due to the use of PS80 (98), as defined herein. The term “stable” means that said preparations remain practically free from visible particles until the end of their authorized shelf life. In one aspect the present preparations remains free of visible particles for up to 36 months, or 30 months; or for up to 24 months; or for up to 18 months. The stability of parenteral protein preparations can be affected by parameters well known to the skilled person, such as light (UV radiation), temperature and/or shaking. Therefore, in one aspect, the term “stable” includes conditions usually recommended for storage of a product comprising the present parenteral protein-, or antibody preparation as, for example, described in the Summary of Product Characteristics (SmPC) issued by the European Medicines Agency (EMA) or the package insert for that given product. In one embodiment, the term “stable” includes a storage period of up to 36 months, or up to 30 months, at a storage temperature between 2° C.-8° C. and substantially protected from light. In another embodiment, the term “stable” includes a storage period of up to 36 months, or up 10 to 30 months, at a storage temperature of about 5° C.

The term “practically free from particles” (pffp) in accordance with the present invention is used for samples with a maximum of 2 visible particles per investigated container, such as e.g. a vial or syringe, and a maximum of 4 visible particles in 10 such containers.

In one embodiment, the term “visible particles” means water insoluble particles comprising one or several types of free fatty acids, or a mixture of fatty acid with polydimethylsiloxane (PDMS), which are detected during visual inspection. In one aspect, types of free fatty acids are those fatty acids which are known to be released from degradation of polysorbates, especially from PS20 or PS80. The visual inspection is a probabilistic method and single visible particles are likely to be detected with a size of at least 100 μm (40%) or 150 μm (70%) (see e.g. James A. Melchore, AAPS PharmSciTech; 2011; 12(1): 215-221; published online on 4 Jan. 2011), whereas the presence of multiple particles in the smaller size range <100 μm may for example be seen as turbidity. Visual inspections can be carried out by methods well known to the person of skill in the art and as, for example, described in European Pharmacopoeia 6.0 (Chapter 2.9.20. “Particulate contamination: visible particles”).

In another embodiment, the present invention provides a pharmaceutical dosage form comprising a preparation as defined herein, for example an aqueous antibody composition, in a container such as, for example, a syringe or vial.

The term “excipient” refers to an ingredient in a pharmaceutical composition or preparation, other than an active ingredient, which is nontoxic to a subject. An excipient includes, but is not limited to, a buffer, stabilizer including antioxidant or preservative.

The term “buffer” is well known to a person of skill in the art of organic chemistry or pharmaceutical sciences such as, for example, pharmaceutical preparation development. Buffer as used herein means acetate, succinate, citrate, arginine, histidine, phosphate, Tris, glycine, aspartate, and glutamate buffer systems. Furthermore, within this embodiment, the histidine concentration of said buffer is from 5 to 50 mM. Preferred buffers are free histidine base and histidine-HCl or acetate or succinate and/or aspartate. Furthermore, within this embodiment, the histidine concentration of said buffer is from 5 to 50 mM.

The term “stabilizer” is well known to a person of skill in the art of organic chemistry or pharmaceutical sciences such as, for example, pharmaceutical preparation development. A stabilizer in accordance with the present invention is selected from the group consisting of sugars, sugar alcohols, sugar derivatives, or amino acids. In one aspect the stabilizer is (1) sucrose, trehalose, cyclodextrines, sorbitol, mannitol, glycine, or/and (2) methionine, and/or (3) arginine, or lysine. In still another aspect, the concentration of said stabilizer is (1) up to 500 mM or (2) 5-25 mM, or/and (3) up to 350 mM, respectively

The term “protein” as used herein means any therapeutically relevant polypeptide. In one embodiment, the term protein means an antibody. In another embodiment, the term protein means an immunoconjugate.

The term “antibody” herein is used in the broadest sense and encompasses various antibody classes or structures, including but not limited to monoclonal antibodies, polyclonal antibodies, multispecific antibodies (e.g., bispecific antibodies), and antibody fragments so long as they exhibit the desired antigen-binding activity. In one embodiment, any of these antibodies is human or humanized. In one aspect, the antibody is selected from alemtuzumab (LEMTRADA®), atezolizumab (TECENTRIQ®), bevacizumab (AVASTIN®), cetuximab (ERBITUX®), panitumumab (VECITIBIX®), pertuzumab (OMNITARG®, 2C4), trastuzumab (HERCEPTIN®), tositumomab (Bexxar®), abciximab (REOPRO®), adalimumab (HUMIRA®), apolizumab, aselizumab, atlizumab, bapineuzumab, basiliximab (SIMULECT®), bavituximab, belimumab (BENLYSTA®) briankinumab, canakinumab (ILARIS®), cedelizumab, certolizumab pegol (CIMZIA®), cidfusituzumab, cidtuzumab, cixutumumab, clazakizumab, crenezumab, daclizumab (ZENAPAX®), dalotuzumab, denosumab (PROLIA®, XGEVA®), eculizumab (SOLIRIS®), efalizumab, epratuzumab, erlizumab, emicizumab (HEMLIBRA®), felvizumab, fontolizumab, golimumab (SIMPONI®), ipilimumab, imgatuzumab, infliximab (REMICADE®), labetuzumab, lebrikizumab, lexatumumab, lintuzumab, lucatumumab, lulizumab pegol, lumretuzumab, mapatumumab, matuzumab, mepolizumab, mogamulizumab, motavizumab, motovizumab, muronomab, natalizumab (TYSABRI®), necitumumab (PORTRAZZA®), nimotuzumab (THERACIM®), nolovizumab, numavizumab, olokizumab, omalizumab (XOLAIR®), onartuzumab (also known as MetMAb), palivizumab (SYNAGIS®), pascolizumab, pecfusituzumab, pectuzumab, pembrolizumab (KEYTRUDA®), pexelizumab, priliximab, ralivizumab, ranibizumab (LUCENTIS®), reslivizumab, reslizumab, resyvizumab, robatumumab, rontalizumab, rovelizumab, ruplizumab, sarilumab, secukinumab, seribantumab, sifalimumab, sibrotuzumab, siltuximab (SYLVANT®) siplizumab, sontuzumab, tadocizumab, talizumab, tefibazumab, tocilizumab (ACTEMRA®), toralizumab, tucusituzumab, umavizumab, urtoxazumab, ustekinumab (STELARA®), vedolizumab (ENTYVIO®), visilizumab, zanolimumab, zalutumumab.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called a, d, e, g, and m, respectively. The light chain of an antibody may be assigned to one of two types, called kappa (κ) and lambda (λ), based on the amino acid sequence of its constant domain.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”). Generally, antibodies comprise six CDRs: three in the VH (CDR-H1, CDR-H2, CDR-H3), and three in the VL (CDR-L1, CDR-L2. CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “pharmaceutical composition” or “pharmaceutical preparation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or preparation, other than an active ingredient, which is nontoxic to a subject. A pharmaceutically acceptable carrier includes, but is not limited to an excipient as defined herein.

A. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al.,

Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

B. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat.

Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boemer et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

C. Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

D. Immunoconjugates

The invention also provides immunoconjugates comprising an antibody herein conjugated (chemically bound) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

E. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172. WO 2010/145792, and WO 2013/026831. The bispecific antibody or antigen binding fragment thereof also includes a “Dual Acting FAb” or “DAF” comprising an antigen binding site that binds to two different antigens, or two different epitopes of the same antigen (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

F. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see. e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, N.J. (2003), pp. 245-254, describing expression of antibody fragments in E. coli.)

After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7): human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W 138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268.

The invention will now be further illustrated by the following, non-limiting working examples.

### Examples

### Material and Methods

Test Antibody Formulation

The antibody tocilizumab was provided by Chugai Pharmaceutical Co., Ltd. The following aqueous antibody formulation was prepared (at pH 6.0):

180 mg/mL tocilizumab,

20 mmol/L L-histidine/L-histidine hydrochloride monohydrate,

100 mmol/L L-Arginine/L-Arginine hydrochloride,

30 mmol/L L-methionine,

0.2 mg/mL PS80 (from either NOF or SEPPIC)

The formulation comprising PS80 (SEPPIC) is also designated as Lot No. S7D01, and the formulation comprising PS80 (NOF, PS80(98)) is designated Lot No. S7D02 (see Table 2, below)

Primary Packaging Material

The test antibody formulation was filled into 1 mL staked-in needle glass syringes equipped with a Plunger stopper. Such glass syringes are for example provided by Nuova Ompi (Syringe: 1.0 mL long, borosilicate glass syringe, type 1, staked-in needle: 27G×½″ Cannula); equipped with a Plunger stopper by Daikyo Seiko (RSH 6.75F RSV D 777-7 RB2-40;)

Stability Program

Stability program, storage condition, and the time points for inspection for each test antibody formulation are shown in Table 1. Samples were stored horizontally protected from light.

TABLE 1
Stability program

| | Month(s) | | | | | | | | | | | | | |
|---|---|---|---|---|---|---|---|---|---|---|---|---|---|---|
| Temperature/RH | 0 | 1 | 2 | 3 | 6 | 9 | 12 | 18 | 21 | 24 | 27 | 30 | 33 | 36 |
| 5° C. | x | — | — | x | x | x | x | x | x | x | x | x | x | x |
| 25° C./60% RH | — | — | — | x | x | — | x | — | — | — | — | — | — | — |
| 40° C./75% RH | — | x | x | x | — | — | — | — | — | — | — | — | — | — |

Evaluation Methods

### Visual Inspection:

Inspections for visible particles was carried our according to European Pharmacopoeia 6.0 (Chapter 2.9.20. “Particulate contamination: visible particles”).

### Particle Identification:

The identification of visible particles was carried out subsequent to their isolation using Fourier-Transform Infrared (FT-IR) spectroscopy.

### Procedure for Isolating Visible Particles:

1. A plunger stopper of a syringe was removed by pulling a plunger rod slowly.
2. Solution containing visible particles was sampled from the syringe, and the solution was put on a clean petri dish.
3. Pictures of the particles were taken with digital microscope VHX-2000 from KEYENCE.
4. The visible particles were put on nickel filter
5. The particles on the filter were rinsed with cold water.
6. Pictures of the particles were taken with digital microscope (model as in step 3).

FT-IR spectroscopy was performed on an IR PRESTIGE-21 from Shimadzu, with the following conditions

| Measurement parameter | Setting value |
|---|---|
| Wavelength range | 4000 cm⁻¹-700 cm⁻¹ |
| Resolution | 4 cm⁻¹ |
| Measurement mode | Specular reflection |

### Results

After storage at 25° C. and 40° C. for 12 and 3 months, respectively, both Seppic and NOF samples remained practically free from visible particles. For storage at 5° C., a lot of visible particles were observed in SEPPIC samples at 24 months, and the number of visible particles in those samples markedly increased with longer storage time beyond 24 months. Particle identification using FT-IR was performed at 24, 30 and 36 months and the particle nature was identified as free fatty acid. In contrast, NOF samples remained practically free of visible particles from initial to 36 months. The summary of observed visible particles is provided in Table 2.

TABLE 2
Summary of observed visible particles

| | | | S7D01 (SEPPIC) | | S7D02 (NOF) | |
|---|---|---|---|---|---|---|
| Storage | | | VP per | VP per 10 | VP per | VP per 10 |
| condition | Storage time | | syringe | syringes | syringe | syringes |
| na | initial | | pffp | pffp | pffp | pffp |
| 5° C. | 3 | months | pffp | pffp | pffp | pffp |
| | 6 | months | pffp | pffp | pffp | pffp |
| | 9 | months | pffp | pffp | pffp | pffp |
| | 12 | months | pffp | pffp | pffp | pffp |
| | 18 | months | pffp | pffp | pffp | pffp |
| | 21 | months | pffp | pffp | pffp | pffp |
| | 24 | months | 3 | 15 | pffp | pffp |
| | 27 | months | 8 | 39 | pffp | pffp |
| | 30 | months | ≥5 | ≥50 | pffp | pffp |
| | 33 | months | ≥5 | ≥50 | pffp | pffp |
| | 36 | months | ≥5 | ≥50 | pffp | pffp |
| 25° C./ | 3 | months | pffp | pffp | pffp | pffp |
| 60% RH | 6 | months | pffp | pffp | pffp | pffp |
| | 12 | months | pffp | pffp | pffp | pffp |
| 40° C./ | 1 | month | pffp | pffp | pffp | pffp |
| 75% RH | 2 | month | pffp | pffp | pffp | pffp |
| | 3 | months | pffp | pffp | pffp | pffp |

pffp means practiclly free from particles; pffp is used for samples with a maximum of 2 visible particles per syringe and a maximum of 4 visible particles in 10 syringes

1. A stable aqueous composition comprising a protein, one or more pharmaceutically acceptable excipient and PS80 (98).

2. The stable aqueous composition according to claim 1, wherein the PS80 (98) is present in a concentration of 0.01 to 1% (w/v); or from 0.01 to 0.06% (w/v); or from 0.02 to 0.05% (w/v); or at about 0.02% (w/v).

3. The stable aqueous composition according to claim 1, wherein the protein is an antibody.

4. The stable aqueous composition according to claim 3, wherein the antibody is tocilizumab.

5-9. (canceled)

10. A method of preventing formation of visible particles in a parenteral protein preparations, the method comprising adding PS80 (98) to a protein composition susceptible to visible particles formation.

11. (canceled)

12. The method according to claim 10, wherein the PS80 (98) is present in a concentration of 0.01 to 1% (w/v), or from 0.01 to 0.06% (w/v), or from 0.02 to 0.05% (w/v), or at about 0.02% (w/v).

13. The method according to claim 10, wherein the parenteral protein preparation remains stable for up to 30 months, or up to 36 months.

14. The stable aqueous composition according to claim 1, which is a parenteral protein preparation.

15. The stable aqueous composition according to claim 1, which remains stable for up to 30 months, or up to 36 months.
